(19) United States
(12) Patent Application Publication
(10) Pub. No.: US 2015/0329579 A1
(43) Pub. Date: Nov. 19, 2015

(54) METHOD FOR MAKING N-SUBSTITUTED MANNOSAMINE DERIVATIVES

(71) Applicant: GLYCOM A/S, Kongens Lyngby (DK)

(72) Inventors: Ioannis VRASIDAS, Thessaloniki (GR); Gábor VLÁD, Budapest (HU); Gyula DEKANY, Sinnamon Park (AU)

(73) Assignee: GLYCOM A/S, Kgs, Lyngby (DK)

(21) Appl. No.: 14/651,573

(22) PCT Filed: Dec. 20, 2013
(86) PCT No.: PCT/EP2013/077628

§ 371 Date: Jun. 11, 2015

(30) Foreign Application Priority Data
Dec. 21, 2012 (EP) 12198918.0

(51) Int. Cl. C07H 5/06

(52) U.S. Cl. CPC C07H 5/06

(57) ABSTRACT

A method for making an N-substituted D-mannosamine derivative of the following formula 1 and its salts

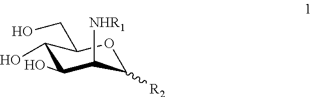

wherein R₁ is a non-electron-withdrawing group, and wherein R₂ is —OH or R₂ is —NHR₃ wherein R₃ is a group removable by hydrogenolysis;

by epimerizing an N-substituted D-glucosamine derivative of the following formula 2

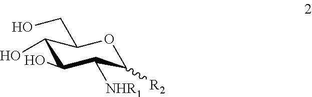

wherein R₁ and R₂ are as defined above.

### FIELD OF THE INVENTION

The present invention relates to a novel method for making N-substituted D-mannosamine derivatives from N-substituted D-glucosamine derivatives.

### BACKGROUND OF THE INVENTION

2-Amino-2-deoxy-D-mannose (D-mannosamine) mainly in its N-acetylated form (ManNAc) can be found as a building block of certain bacterial capsular polysaccharides and lipopolysaccharides. In addition, N-acetyl-D-mannosamine is the biosynthetic precursor of sialic acid, a unique nine-carbon ketoaldonic acid having many major biological roles.

One can obtain D-mannosamine or N-acetyl-D-mannosamine by chemical or enzymatic transformations. N-Acetyl-D-glucosamine can be converted into N-acetyl-D-mannosamine via 2-epimerization which can be initiated with bases (organic or inorganic bases as well as basic ion exchange resins are suitable, see e.g. EP-A-385287 or WO 2007/135086) or epimerase enzymes (Yamaguchi et al. Trends Glycosci. Glycotechnol. 18, 245 (2006)). In these methodologies, an equilibrium between N-acetyl-D-glucosamine and N-acetyl-D-mannosamine is formed wherein the gluco compound is favoured, thus sophisticated and/or complicated separation techniques are needed to isolate this minor product from the starting material remaining, reagents and/or enzymes and other undesired by-products. The isolation difficulties, the moderate chemical yield and the relative expense of the starting compound all prevent this procedure from being scaled-up to provide a cost-effective industrial process.

Kuhn et al. (Liebigs Ann. Chem. 628, 172 (1959)) reported that 2-benzylamino-2-deoxy-D-gluconic acid nitrile and 2-benzylamino-2-deoxy-D-mannonic acid nitrile, respectively, could be epimerized in boiling methanol to the same mixture containing the compounds in a ratio of about 1:1.

The biological importance of mannosamine derivatives has always been an incentive for developing new, shorter and simpler synthetic methods for making them on an industrial scale (see e.g. WO 2012/140576). It is an aim of the present invention to provide such a method.

### SUMMARY OF THE INVENTION

This invention relates to a method for making an N-substituted D-mannosamine derivative of the following formula 1 and its salts

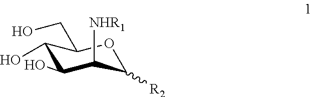

wherein R₁ is a non-electron-withdrawing group, and wherein R₂ is —OH or

R₂ is —NHR₃ wherein R₃ is a group removable by hydrogenolysis;

by epimerizing an N-substituted D-glucosamine derivative of the following formula 2

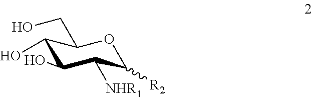

wherein R₁ and R₂ are as defined above.

Preferably, each of the R₁ and R₃ groups removable by hydrogenolysis is a benzyl or naphthylmethyl group optionally substituted with one or more phenyl, alkyl or halogen groups, more preferably a benzyl group.

### DETAILED DESCRIPTION OF THE INVENTION

In accordance with this invention, the term “group removable by hydrogenolysis” preferably means a protecting group whose bond attached to the core carbohydrate structure can be cleaved by addition of hydrogen in the presence of catalytic amounts of palladium, Raney nickel or another appropriate metal catalyst known for use in hydrogenolysis, resulting in the regeneration of the protected functional group, mainly —OH or —NH₂ of the parent molecule. Such protecting groups are well known and are thoroughly discussed in P. G. M. Wuts and T. W. Greene: Protective Groups in Organic Synthesis, John Wiley & Sons (2007).

Suitable protecting groups include, but are not limited to, benzyl, diphenylmethyl (benzhydryl), 1-naphthylmethyl, 2-naphthylmethyl or triphenylmethyl (trityl) groups, each of which can optionally be substituted by one or more substituent groups selected from: alkyl, alkoxy, phenyl, amino, acylamino, alkylamino, dialkylamino, nitro, carboxyl, alkoxycarbonyl, carbamoyl, N-alkylcarbamoyl, N,N-dialkylcarbamoyl, azido, halogenalkyl or halogen. Preferably, such substituent groups, if present, are on the aromatic ring(s). Preferably, a group removable by hydrogenolysis is a benzyl group optionally substituted by alkyl, alkoxy, nitro or halogen.

Herein, the term “non-electron-withdrawing group” preferably means a hydrogen atom or a carbon-bonded electron-donating group selected from: the alkyl, cycloalkyl, aralkyl (such as benzyl or a group removable by hydrogenolysis defined above), alkenyl, cycloalkenyl, alkynyl and aromatic groups, all of which can be substituted by an alkyl, alkoxy, phenyl, amino, acylamino, alkylamino, dialkylamino, nitro, carboxyl, alkoxycarbonyl, carbamoyl, N-alkylcarbamoyl, N,N-dialkylcarbamoyl, azido, halogenalkyl and/or halogen. Preferably, the non-electron-withdrawing group is an electron-donating group, and more preferably a group removable by hydrogenolysis.

This invention provides a method for making an N-substituted D-mannosamine derivative of formula 1 and its salts

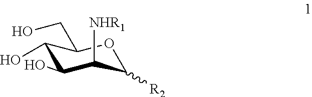

wherein R₁ is a non-electron-withdrawing group, and wherein R₂ is —OH or

R₂ is —NHR₃ wherein R₃ is a group removable by hydrogenolysis;

by epimerizing an N-substituted D-glucosamine derivative of formula 2 or its salt:

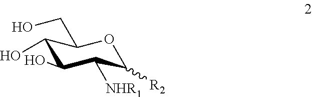

wherein R₁ and R₂ are as defined above.

The preferred R₁-group in compounds of formula 1 and 2 is H or a group removable hydrogenolysis, more preferably a benzyl or naphthylmethyl group optionally substituted with one or more groups selected from phenyl, alkyl or halogen. Even more preferably, R₁ is benzyl. These preferred protecting groups have the advantage that the by-products of their hydrogenolysis are exclusively toluene, methylnaphthalene, or substituted toluene or methylnaphthalene derivatives, respectively. Such by-products can easily be removed even in multi-ton scales from water soluble saccharide products via evaporation and/or extraction processes. The preferred R₂-group is —OH.

The method of epimerizing the N-substituted D-glucosamine derivative of formula 2, preferably its salt, in accordance with this invention to produce the N-substituted D-mannosamine derivative of formula 1 can be carried out with a calcium-ion containing substance in the presence of a base. A calcium-ion containing substance preferably means inorganic or organic calcium compounds such as calcium salts (e.g. chloride, carbonate, bicarbonate, sulfate, phosphate, acetate), calcium hydroxide, calcium oxide, calcium containing minerals (e.g. zeolites, apatites) or even ion exchange resins in Ca²⁺-form. The epimerization reaction can be suitably carried out in a protic solvent or in a mixture of protic solvents. The protic solvent can be water, acetic acid or a C₁-C₆ alcohol. A mixture of one or more protic solvents with one or more suitable aprotic organic solvents partially or fully miscible with the protic solvent(s) (such as THF, dioxane, ethyl acetate or acetone) can also be used. Water, one or more C₁-C₆ alcohols, or a mixture of water and one or more C₁-C₆ alcohols are preferably used as the solvent system, particularly a methanol/water mixture. The base used in the epimerization reaction shall be applied to make the pH of the reaction milieu basic, preferably not less than 10. The base of choice is generally a strong base, e.g. LiOH, NaOH, KOH, Ba(OH)₂, Ca(OH)₂, K₂CO₃, basic ion exchange resins in OH⁻-form, tetraalkylammonium hydroxides, etc. The reaction mixture should be vigorously agitated, at a temperature of 20-60° C. Preferably, the temperature is kept at around 20-25° C. until the conversion reaches about 40-60%, then at around 40-50° C. to complete the reaction.

Without wishing to be bound by any theory, it is believed that the base seems to play a double role in the epimerization reaction of this invention. First, if the N-substituted D-glucosamine derivative of formula 2 is provided as a salt, it neutralizes that salt. Second, it causes the epimerization under the basic reaction conditions while the Ca²⁺-ion creates a complex with the N-substituted D-glucosamine derivative of formula 2.

In a preferred embodiment, the base and the calcium-ion containing substance is the same, and it is selected from calcium oxide, calcium carbonate, calcium hydroxide and any mixture thereof, most preferably calcium hydroxide. At least 1 equivalent (1 mole to 1 mole of the N-substituted D-glucosamine derivative of formula 2) of calcium hydroxide should be provided for the neutralization of the salt, preferably the hydrochloride salt, of the compound of formula 2, and some additional calcium hydroxide is provided for the epimerization itself Preferably, at least 1.5 equivalents, more preferably at least 2.5-3 equivalents of calcium hydroxide are used. Although greater amounts of calcium hydroxide increase yield, the calcium hydroxide needs to be removed after the reaction takes place. For this reason, it is preferred to use no more than about 3 equivalents of calcium hydroxide.

The calcium ion is preferably removed from the reaction mixture by precipitation. Preferred anions that form precipitates with calcium are carbonate, sulfate and oxalate, preferably sulfate, resulting from a treatment with sulfuric acid. In this regard, the sulfuric acid neutralizes the base, which is preferably calcium hydroxide. The resulting precipitated calcium salt can be removed from the reaction mixture by conventional filtration methods.

Preferably, the N-substituted D-mannosamine derivative of formula 1, wherein R₁ and R₃, if present, are groups removable by hydrogenolysis, is then subjected to hydrogenolysis to form free D-mannosamine. The hydrogenolysis can be carried out as described in WO 2012/140576 to remove the R₁ and optionally R₃ groups. The D-mannosamine and D-glucosamine (coming from the non-epimerized starting material of formula 2) in the resulting reaction mixture can be converted into their respective acid addition salts in a conventional manner, using an inorganic or organic acid or salt as described in WO 2012/140576. The mixture of the salts of D-mannosamine and D-glucosamine can then be separated in a conventional manner by selective crystallization as described in WO 2012/140576 to yield a pure D-mannosamine salt. The D-mannosamine salt can be used for making useful mannosamine containing oligo- and polysaccharides, or N-acetylmannosamine which can then be transformed to neuraminic acid and its derivatives as described in WO 2012/140576.

A compound of formula 2 wherein R₂ is —OH can be made by a process as described in WO 2012/140576, wherein:
a) fructose is reacted with an excess of a primary amine, R₁-NH₂, to yield a fructosyl amine derivative;
b) the fructosyl amine derivative is isolated as a crude product by separating excess R₁-NH₂ from it; and then
c) the crude product of step (b) is treated with an acid to obtain the compound of formula 1 wherein R₂ is OH by a Heyns-rearrangement.

A compound of formula 2 wherein R₂ is —NHR₃ can also be made by a process as described in WO 2012/140576, wherein:
a) fructose is reacted with an excess of a primary amine, R₁-NH₂, and a salt of the amine, and then
b) the compound of formula 1, wherein R₂ is —HR₃, is separated from the reaction mixture resulting from step (a).

A compound of formula 2, wherein R₂ is —HR₃, can be easily converted into a compound of formula 2, wherein R₂ is —OH, by treatment with an acid to remove the acid labile —NHR₃ group and regenerate the anomeric OH as described in WO 2012/140576.

### EXAMPLE

A solution of N-benzyl-2-amino-2-deoxy-D-glucose hydrochloride (60 g) in a mixture of methanol and water (480 ml, 2:1 volumetric mixture) with 43.7 g of calcium hydroxide is stirred at 25° C. for 20 hours. The temperature was then increased to 40° C. and the suspension was stirred for a further 20 hours. A sample of the reaction mixture was analysed by TLC and showed ca. 10:1 ratio of N-benzyl-2-amino-2-deoxy-D-mannose to N-benzyl-2-amino-2-deoxy-D-glucose. The solution was cooled to 0° C., and the reaction mixture was treated dropwise with ˜10 w/w % sulfuric acid until pH became 3.5-4. The resulting suspension was then filtered through a G3 glass filter and concentrated to ca. 150 ml by removing the methanol and part of the water by evaporation.

The remainder of the suspension was diluted to 300 ml with methanol, 3.0 g charcoal on palladium (10 w/w %) was added, and the mixture was subjected to hydrogenolysis at 4-7 bar pressure at 40° C. The reaction time was 12 hours. The mixture was then filtered through a G4 glass filter and concentrated to ca. 50-60 ml by removing the methanol and a part of the water by evaporation. The resulting very thick oil was added dropwise to 300 ml of vigorously stirred ethanol, and the precipitated solid was removed by filtration after 6 hours of stirring at 20° C. The water content of the mother liquor was removed by azeotropic distillation with ethanol, and the finally obtained ca. 50-60 ml suspension was diluted to 250 ml with ethanol and stirred 18 hours at 20° C. The resulting suspension was filtered, washed with ethanol and 2-propanol, and dried in vacuo (35° C., 10 mbar) to yield 10-15 g of mannosamine hydrochloride containing ca. 6% of glucosamine hydrochloride (determined by HPLC).

1. A method for making an N-substituted D-mannosamine derivative of formula 1 or a salt thereof 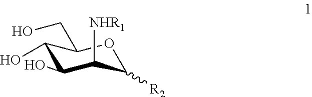
wherein R₁ is a non-electron-withdrawing group, and wherein R₂ is —OH or R₂ is —NHR₃ wherein R₃ is a group removable by hydrogenolysis;
by epimerizing an N-substituted D-glucosamine derivative of formula 2 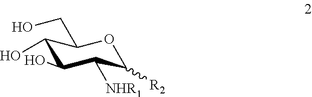
wherein R₁ and R₂ are as defined above.

2. The method of claim 1, wherein the epimerization is carried out with a calcium-ion containing substance in the presence of a base.

3. The method of claim 2, wherein Ca(OH)₂ is used as both the calcium-ion containing substance and the base.

4. The method of claim 3, wherein the treatment with Ca(OH)₂ is carried out at a temperature of 20-60° C.

5. The method of claim 3, wherein the treatment with Ca(OH)₂ is in a protic solvent or in a mixture of protic solvents.

6. The method of claim 1, wherein R₁ is a group removable by hydrogenolysis and R₂ is —OH.

7. The method of claim 6, wherein the N-substituted D-mannosamine derivative of formula 1 is subjected to catalytic hydrogenolysis to make D-mannosamine or a salt thereof.

8. The method of claim 1, further comprising the step of Heyns rearrangement of fructose to produce the N-substituted D-glucosamine derivative of formula 2.

9. The method of claim 4, wherein the treatment with Ca(OH)₂ is carried out at a temperature of 40-50° C.

10. The method of claim 6, wherein R₁ is a benzyl or naphthylmethyl group optionally substituted with one or more phenyl, alkyl or halogen groups.

11. The method of claim 10, wherein R₁ is a benzyl group.

12. The method of claim 4, wherein the treatment with Ca(OH)₂ is in a protic solvent or in a mixture of protic solvents.
